(19) United States
(12) Patent Application Publication
(10) Pub. No.: US 2010/0068739 A1
(43) Pub. Date: Mar. 18, 2010

(54) PH Tolerant Luciferase

(76) Inventors: Laurence Tisi, Cambridge (GB); Gim Hoong Erica Law, Cambridge (GB); Olga Gandelman, Cambridge (GB); James Augustus Henry Murray, Cambridge (GB)

Correspondence Address: NIXON & VANDERHYE, PC, 901 NORTH GLEBE ROAD, 11TH FLOOR, ARLINGTON, VA, 22203, US

(21) Appl. No.: 11/990,228

(22) PCT Filed: Aug. 9, 2006
(86) PCT No.: PCT/GB2006/002974

§ 371 Date: Feb. 8, 2008

(30) Foreign Application Priority Data
Aug. 10, 2005 (GB) 0516455.3

(51) Int. Cl. C12Q 1/66; C12N 9/02

(52) U.S. Cl. 435/8; 435/189

(57) ABSTRACT

Use of a luciferase that has a mutation of at least one amino acid selected from the group consisting of positions 14, 35, 182, 232 and 465, where the numbering is according to the sequence of the luciferase from P. pyralis (SEQ ID NO:1) in a method that is performed at a pH below the optimal pH for the wild-type luciferase during at least part of the time period over which bioluminescence measurements are taken, wherein the specific activity of the mutant luciferase is higher than the specific activity of wild-type at the pH at which the method is carried out.

### FIELD OF THE INVENTION

The present invention relates to the use of a pH tolerant luciferase in a method that is performed below the optimal pH of a luciferase.

### BACKGROUND OF THE INVENTION

Firefly luciferase catalyses the efficient transfer of chemical energy into light via a two-step process, using ATP-Mg²⁺, firefly luciferin and molecular oxygen (DeLuca, M., (1976), “Firefly Luciferase”, Advances in enzymology and related areas of molecular biology, 44, 37-68):

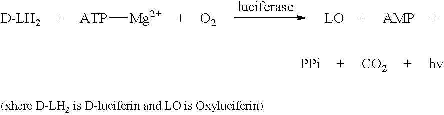

Various studies have genetically altered recombinant wild-type luciferases, in order to obtain enzymes that are more useful in luminometric methods. This work has generally focused on the instability of luciferase when used or stored at high temperatures, for example, in excess of 30° C. For example, WO 01/20002 discloses various examples of luciferase enzymes having increased thermostability.

It is also known that firefly luciferase from Photinus pyralis and related firefly luciferases are pH-sensitive and that this pH sensitivity can lead to reduced detectable light signals in non-optimum pH conditions. The optimal pH for bioluminescence has been reported to be between 7.7 and 8.1 depending on the exact instrumentation and buffer systems (Green, A. A. and W. D. McElroy (1956), “Crystalline firefly luciferase”, Biochim. Biophys. Acta 20, 170-176; Dementieva, E. I., L. Y. et al., (1986), “pH dependence of the bioluminescence spectra and kinetic constants of luciferase of the firefly Luciola mingrelica”, Biochemistry (Moscow) 51, 130-139; Thompson, J. F. et al., (1997), “Mutation of a protease-sensitive region in firefly luciferase alters light emission properties”, J. Biol. Chem. 272, 18766-18771). The efficiency of light emission is measured in terms of quantum yield, which is defined as the number of photons emitted per molecule of luciferin consumed. At pH levels above 7.5, the quantum yield is around 0.88. In contrast, the quantum yield at pH 6.0 is only around 0.5, hence less light is emitted by firefly luciferases at pH 6.0 than at above pH 7.5 (Seliger, H. H. and W. D. McElroy (1959), “Quantum yield in the oxidation of firefly luciferin”, Biochem. Biophys. Res. Commun. 1, 21-24; Seliger, H. H. and W. D. McElroy (1960), “Spectral emission and quantum yield of firefly bioluminescence”, Arch. Biochem. Biophys. 88, 136-141).

Most firefly luciferases, such as the luciferase from Photinus pyralis, demonstrate pH dependent bathochromic shifts (i.e. red-shifts) of their bioluminescent spectra as the pH of the reaction is dropped from pH 8.0 to pH 6.0. As the pH is decreased from pH 8.0 to pH 6.0, the emitted light changes from yellow-green to red.

Red light emission is proposed to arise from excited keto-oxyluciferin, which exists in tautomeric equilibrium in the excited state with its enolic form, a yellow-green emitter (White, E. H. et al., “Chemi- and bioluminescence of firefly luciferin”, J. Am. Chem. Soc., 91, 2178-2180, 1969; White, E. H. and Roswell D. F., “Analogs and derivatives of firefly oxyluciferin, the light emitter in firefly bioluminescence”, Photochem. Photobiol., 53, 131-136, 1991; Fukushima, K. “Information on the chemiluminescence mechanism of the firefly from MNDO molecular orbital calculations”, J. Mol. Struc.: Theochem., 235, 11-14, 1991). The emission colour may be determined by kinetic competition between enolization and radiative deactivation of the primarily formed singlet keto-oxyluciferin.

Bathochromic shifts observed in both native and recombinant wild-type firefly luciferases have been investigated extensively. It has been generally accepted that the shift occurs due to a change in enzyme configuration (see e.g., Seliger “The colors of firefly bioluminescence: Enzyme configuration and species specificity”, 1964, Proc. Natl. Acad. Sci. USA., 52, 75-81; Viviani, “Bioluminescence of Brazilian fireflies (Coleoptera: lampyridae): spectral distribution and pH effect on luciferase-elicited colors. Comparison with elaterid and phengodid luciferases”, 1995, Photochem. Photobiol. 62, 490-495). Mutation studies on L. cruciata luciferase showed that the colour of emitted light can be changed by the mutation of just a single amino acid residue (Kajiyama, N. and Nakano, E. (1991) “Isolation and characterization of mutants of firefly luciferase which produce different colours of light”, Prot. Eng., 4, 691-693).

Investigation of the mechanism behind bathochromic shifts have largely been focussed on the enzyme's active site where mutation of residues in the luciferase active site showed an alteration to the emission spectra (Branchini, B. R. et al., (1998), “Site-directed mutagenesis of histidine 245 in firefly luciferase: A proposed model of the active site”, Biochemistry, 37, 15311-15319; Branchini, B. R. et al., (1999), “Site-directed mutagenesis of firefly luciferase active site amino acids: A proposed model for bioluminescence colour”, Biochemistry, 38, 13223-13230; Branchini, B. R. et al., (2001), “The role of active site residue arginine 218 in firefly luciferase bioluminescence”, Biochemistry, 40, 2410-2418).

Recent results have additionally suggested that the 2′ hydroxyl of the ribose moiety of 2′ deoxyATP plays a role in stabilising the conformation of the enzyme required for green bioluminescence, again supporting the hypothesis that the conformation of the enzyme's active site determines the bathochromic shift (Tisi, L. C. et al., “The basis of the bathochromic shift in the luciferase from Photinus pyralis”, Bioluminescence and Chemiluminescence: Progress and Current Applications, 2002, 57-60).

Bathochromic shifts have a considerable negative impact on the utility of firefly luciferases in a whole range of applications. For example, luciferase/luciferin assay systems generally use standard photomultiplier tubes to detect the light produced by the luciferase. However, as standard photomultiplier tubes are less sensitive to red light than to yellow-green light, the bathochromic shift is an undesirable trait for a luciferase used in a method performed at acidic pH, or in which pH fluctuates.

At pH values below the optimal pH for firefly luciferases, aside from the colour of emitted light being red-shifted, the total amount of light emitted decreases (Seliger, H. H. and W. D. McElroy (1959)). This also adversely affects numerous applications of firefly luciferases where assays may be performed at sub-optimal pH or where decreases in pH may be encountered. This has particular relevance for methods where a recombinant luciferase is used as a reporter gene in vivo, in particular for in vivo imaging. For example, where the gene for wild-type Photinus pyralis luciferase is used as a reporter for the imaging of tumours in animal models, the imaging of the tumour can be adversely affected if the tumour cell's intracellular environment becomes acidified (as can commonly occur) since the luciferase will emit less light as the pH decreases. As such, the imaging of the tumour may become unreliable, difficult or impossible, if there is a reduction in the intracellular pH of the tumour cells. This issue is of particular significance for studies on tumour cells which are growing under conditions where their intra-cellular pH is lower (e.g. in cases where tumour cells are relying disproportionately on glycolysis: a major feature of metastasis research (Schornack P. A. and Gillies R. J., (2003), “Contributions of metabolism and H+ diffusion to the acidic pH of tumors”, Neoplasia, 5: 135-145).

In the aforementioned example, it would be advantageous to use a luciferase that emits the same amount of light regardless of pH, such that variations in intracellular pH would not adversely effect the ability to image the tumour. Whilst no such luciferase variant exists (or could be expected to exist), mutant luciferases have been identified whose light emitting properties are less affected by reductions in pH relative to their wild-type equivalent (see below).

Additionally, since in vivo imaging is commonly performed in mammalian animal models where the temperature of the animal will generally be greater than 30° C. and most likely at 37° C., the luciferase used for imaging should preferably be thermostable, in that its half-life at temperatures above 30° C., or preferably at 37° C., is greater than the recombinant wild-type equivalent. This is because the bioluminescent signal obtained during imaging will be greatly reduced if the luciferase used becomes rapidly inactivated due to the elevated temperature in vivo. In fact it has been proven that thermostable luciferases greatly improve the imaging of tumours in animal models (Baggett B. et al., (2004), “Thermostability of Firefly Luciferases Affects Efficiency of Detection by In Vivo Bioluminescence”, Molecular Imaging, Vol. 3 No. 4, 324-332).

However, neither ‘pH tolerance’ nor ‘thermostability’ per se are sufficient for a luciferase mutant to have improved utility for in vivo imaging applications: the luciferase mutant must also emit sufficient light to be sensitively detected and hence imaged. This point is relevant as a number of mutant luciferases (as described below) with increased pH tolerance or increased thermostability, emit far less light than the recombinant wild-type enzyme under optimal conditions.

Hence a preferred luciferase for in vivo imaging will have, at least, three key properties:

a) improved tolerance to pH values below the optimum pH for firefly luciferases,
b) improved thermostability; and
c) no significant decrease in the maximal amount of light emitted, relative to wild-type luciferases, under optimal conditions.

A number of recombinant luciferases that have increased tolerance to acidic pH are known in the art. For example, US 2003/0232404 describes luciferases that have increased stability at pH 4.5. Clone 49-7C6 has the following mutations: E2A, L92I, N184Y, H221L, C222A, T250M, A263V, F295L, D354N, T355N, T387P, S400G, K547T, S548N and K549G; Clone 78-0B10 has the following mutations: E2A, Y28D, L92V, Y145S; 1174S, N184Y, S205P, H221L, C222A, T250M, A263V, F295L, D354K, T3550, V357A, T387P, D395A, S400G, N413D, K414N, N500D, K547T, S548N and K549G; Clone 90-1B5 has the following mutations: E2A, A18E, Y28D, S37P, L92V, A102V, S106N, I126V, Y145S, V146I, I174S, N184Y, V1951, V204L, S205P, H221L, C222A, T250M, A263V, F295L, D354K, T355G, V357A, R358K, T387P, D395P, S400G, N413D, K414N, N500D, F501Y, S503A, K547T, S548N and K549G; Clone 133-1B2 has the following mutations: E2A, A18E, Y28D, S37P, G85S, L92V, A102V, S106N, I126V, Y145S, V146I, E156D, I174S, N184Y, V195I, V204L, S205P, H221I, C222A, T235S, T250M, A263V, F295L, D354K, T355G, V357A, R358K, T387P, D395P, S400G, N413D, K414N, N500D, F501Y, S503A, K517I, F539L, K547T, S548N and K549G; Clone 146-1H2 has the following mutations E2A, A18E, Y28D, D35A, S37P, G85S, L92V, A102V, S106N, I126V, Y145S, V146I, I174S, N184Y, L194S, V195I, V204L, S205P, H2211, C222A, T235S, T250M, A263V, F295L, D354K, T355G, V357A, R358K, F368L, T387P, D395P, S400G, N413D, K414N, N500D, F501Y, S503A, F539L, K547T, S548N and K549G. The numbering presented refers to equivalent positions in P. pyralis luciferase, but all “native” amino acids used are from Photuris pennsylvanica luciferase mutant, lucPpe2 (US 2003/0232404), from which the aforementioned mutants were obtained.

The bathochromic shift has been found to be significantly reduced in P. pyralis recombinant luciferase mutants containing one or more of the following mutations: T214A, I232A, F295L and E354K (Tisi, L. C. et al., “The basis of the bathochromic shift in the luciferase from Photinus pyralis”, Bioluminescence and Chemiluminescence: Progress and Current Applications, 2002, 57-60), in L. cruciata containing single amino acid residue substitutions of G326S, H433Y and V239I (equivalent residues in P. pyralis luciferase are 324, 431 and 237 respectively) (Kajiyama, N. and Nakano, E. (1991) “Isolation and characterization of mutants of firefly luciferase which produce different colours of light” Prot. Eng., 4, 691-693.), in E356RN368A mutants of H. parvula (equivalent to positions 354 and 366 in P. pyralis luciferase) (Kitayama, “Creation of a thermostable firefly luciferase with pH-insensitive luminescent color”, 2003) and in a T219I, V239I mutant (Hirokawa K. et al., “Improved practical usefulness of firefly luciferase by gene chimerization and random mutagenesis”, 2002, Biochim Biophys Acta., 1597(2):271-9). Point mutations at D234G, A105V and S420T of the luciferase from Photinus pyralis have also been found to considerably reduce the bathochromic shift (Tisi, L. C. et al., “The basis of the bathochromic shift in the luciferase from Photinus pyralis”, Bioluminescence and Chemiluminescence: Progress and Current Applications, 2002, 57-60). Mutation of T217I in L. cruciata, which corresponds to position 215 of P. pyralis, was shown to increase pH stability and “specific activity” of the enzyme although no results were presented in the literature (Kajiyama, N. and E. Nakano (1993). Thermostabilization of firefly luciferase by a single amino acid substitution at position 217. Biochemistry 32, 13795-13799.). The enzymological term “specific activity”, refers to the amount of a particular enzymatic activity detected per unit time and per unit mass of enzyme under various defined assay conditions (e.g. of pH and temperature) but where the substrates of the enzyme are always present in saturating amounts. Therefore the term “specific activity” as used herein, refers to the amount of light a unit amount of luciferase produces in unit time under defined assay conditions but where ATP and Luciferin are provided at saturating concentrations. However, commonly, the means of light detection used to determine specific activity is less sensitive to red light, as such artificially low specific activities can be obtained for luciferases emitting red light. Thus quoted ‘specific activities’ can, in some cases, be an underestimate. However it is possible to measure luciferase specific activity (using a suitably calibrated spectrophotometer) where the observed specific activity is not affected by the colour of emitted light: herein, where specific activity has been measured in this way we refer to the measurement as “corrected specific activity” rather than simply “specific activity”.

On the whole, the mutations described above confirm thermostability on the luciferase in question (as defined here ‘thermostability’ refers to an increase in the half-life of the luciferase specific activity under given conditions and at a given temperature, for example the half-life at 37° C.). In fact it has generally been found that mutations or conditions (such as stabilising agents or low temperature) that increase the stability of firefly luciferases cause a reduction in the pH dependent bathochromic shift. As previously mentioned, if the apparatus used to measure light emission is less sensitive at detecting red light compared to green light, mutant luciferases that exhibit a reduction in the pH dependent bathochromic shift may appear to have improved specific activity relative to the wild-type equivalent under conditions where the pH is below the optimum of firefly luciferases. As such, in general, thermostable luciferase mutants may have an apparent increased tolerance to conditions where the pH is below the optimum for firefly luciferases as a result of a reduction in the pH dependent bathochromic shift.

However, other effects not directly related to thermostability can also increase the pH tolerance of a firefly luciferase.

Whilst a number of discrete mutations have been demonstrated to affect the performance of recombinant luciferases, in general, any single mutation alone may not confer enough of an effect to provide a mutant firefly luciferase with significantly greater practical utility for a particular application. As a result, it has been common practice to combine a number of favourable mutations in order to gain an additive increase in performance for a particular application. An example of this are the mutants described in US2003/0232404 where as many as 40 mutations are combined in a single mutant. A further example is described by Tisi et al., (“Development of a thermostable firefly luciferase”, Analytica Chimica Acta., 457, (2002), 115-123), where four point mutations were combined with an additive effect on the half-life of the luciferase and its pH tolerance (as subsequently described in Tisi, L. C. et al., “The basis of the bathochromic shift in the luciferase from Photinus pyralis”, Bioluminescence and Chemiluminescence: Progress and Current Applications, 2002, 57-60).

However, whilst properties such as half-life and pH tolerance are additively improved by the combination of several mutations, it has been found that the overall specific activity of the recombinant mutants is reduced as more mutations are added. For example, the mutant Photinus pyralis luciferase described in Tisi et al. (“Development of a thermostable firefly luciferase”, Analytica Chimica Acta., 457, (2002), 115-123) where four point mutations were combined with an additive effect on the half-life of the luciferase and its pH tolerance as shown in Tisi, L. C. et al., “The basis of the bathochromic shift in the luciferase from Photinus pyralis”, Bioluminescence and Chemiluminescence: Progress and Current Applications, 2002, 57-60), whilst having a half-life at 45° C., which is ten times longer than recombinant wild-type Photinus pyralis luciferase (henceforth the wild-type recombinant luciferase from P. pyralis will be referred to here as ‘LucWT’), it only had 50% of the specific activity. Further, the recombinant firefly luciferase mutant with the longest half-life at 45° C. to date, the “UltraGlow’ luciferase from Promega which contains over ten different point mutations, has a specific activity of only 2% of that of His-LucWT under optimal conditions (pH 7.8; table 3).

Hence, whilst desirable properties such as pH resistance can be enhanced by the combination of several point mutations (each conferring a small amount of pH resistance), combining mutations has led to a decrease in the specific activity of the resulting luciferase mutants with the effect that the mutant produces less light than the wild type luciferase under most conditions. This represents a significant problem in assays and other methods where any decrease in the light emitted from a luciferase would adversely affect the performance of the assay or method. This issue is especially serious where recombinant luciferases are used for in vivo imaging since any reduction in the in vivo specific activity of the luciferase being used would decrease the sensitivity of the imaging process.

Clearly, it would be preferable to be able to combine mutations that confer a desirable property such as pH resistance without reducing the specific activity of the resulting luciferase mutant.

The invention provides a method of performing an assay under conditions where the pH is below the optimal pH of firefly luciferases (generally around pH 7.8), or fluctuates to below this pH, in which a luciferase that is more pH tolerant than recombinant wild type luciferase is used but where the specific activity (or corrected specific activity) of the pH tolerant luciferase is similar to, and preferably not legs than, the recombinant wild type luciferase equivalent at the pH optima of wild type luciferase.

### DETAILED DESCRIPTION OF THE INVENTION

The invention provides the use of a luciferase that has a mutation of at least one amino acid selected from the group consisting of positions 14, 35, 182, 232 and 465, where the numbering is according to the sequence of the luciferase from P. pyralis (SEQ ID NO:1-wild type P. pyralis) in a method that is performed at least partly at a pH below the optimal pH for the wild-type version of the luciferase being used during at least part of the time period over which bioluminescence measurements are taken, wherein the specific activity of the mutant luciferase is higher than the specific activity of the wild-type version of the luciferase being used at the pH at which the method is carried out. All amino acid numbering used herein refers to the sequence of P. pyralis, unless otherwise specified.

In one manifestation of the invention, the mutant luciferase is used in an in vivo method. In in vivo methods, the cellular pH is likely to fluctuate, due to for instance, metabolic poisoning and cellular anaerobic respiration. As such, it may not be practical to use luciferase enzymes that are sensitive to pH change. The inventors' finding that a luciferase having a mutation at least one position selected from the group consisting of positions 14, 35, 182, 232 and 465 results in a luciferase that is tolerant to acidic pH makes the use of such a luciferase particularly suitable for this type of method. This is especially the case as none of the individual mutations, nor the combination of all five mutations, has an appreciable negative effect on the specific activity (or corrected specific activity) relative to the wild-type enzyme at optimal pH.

The invention provides for methods where the pH may remain constant throughout or may fluctuate during the method. When the pH remains constant throughout the method, the pH is below the optimal pH for the wild-type version of the luciferase being used. When the pH fluctuates, it may also fluctuate to the optimal pH and/or to alkaline pH, provided that the pH is below the optimal pH for the wild-type version of the luciferase being used during at least part of the time period over which bioluminescence measurements are taken.

Generally, the optimal pH of the wild-type luciferase being used is between pH 7.7 and pH 8.1 and most commonly, the optimal pH is pH 7.8. Thus, the pH is preferably below pH 7.7, more preferably pH 7.6 or below, during at least part of the time period over which bioluminescence measurements are taken. For example, the pH may be within the range of 6.0 to 7.6 during at least part of the time period over which bioluminescence measurements are taken. Even more preferably, the pH is below pH 7.0 during at least part of the time period over which bioluminescence measurements are taken. For example, the pH may be within the range 6.2 to 6.8, 6.3 to 6.7 or 6.4 to 6.6 during at least part of the time period over which bioluminescence measurements are taken. Preferably, the pH is around 6.5 during at least part of the time period over which bioluminescence measurements are taken. Most preferably, the method is performed at pH 6.5 during at least part of the time period over which bioluminescence measurements are taken.

Preferably, the pH is below the optimal pH for the wild-type version of the luciferase for at least 0.1% of the time period over which bioluminescence measurements are taken, for example from between 0.1% and 10%, or between 0.5% and 5% of the time period over which bioluminescence measurements are taken. More preferably, the pH is below the pH optima of the wild-type luciferase for at least 5%, more preferably at least 10%, 20%, 30%, 40%, 50%, 60%, 70%, 80%, 90% or 95% of the time period over which bioluminescence measurements are taken. Thus, throughout the method, or at some point during the method during the time period over which bioluminescence measurements are taken, the pH is preferably below pH 7.7, more preferably pH 7.6 or below, for example within the range of 6.0 to 7.6, 6.2 to 6.8, 6.3 to 6.7 or 6.4 to 6.6. Preferably, the pH is around 6.5 for the whole method or during at least part of the time period over which bioluminescence measurements are taken. Most preferably, the pH is 6.5 for the whole method or during at least part of the time period over which bioluminescence measurements are taken.

Preferably, at least 1%, 5%, 10%, 20%, 30%, 40%, 50%, 60%, 70%, 80%, 90% or 95% of the bioluminescence measurements are taken when the pH is below the optimal pH for the wild-type version of the luciferase being used. Preferably, at least one, two, three, four, five, ten, fifteen, twenty, fifty, one hundred, five hundred, one thousand, two thousand or five thousand bioluminescence measurements are taken when the pH is below the optimal pH for the wild-type version of the luciferase being used. More preferably, all of the bioluminescence measurements are taken when the pH is below the optimal pH for the wild-type version of the luciferase being used.

A luciferase as described above is known in the art (Law, G. H. et al., “Altering the surface hydrophobicity of firefly luciferase”, Abstract published at International Symposium of Bioluminescence and Chemiluminescence, 2002).

Previous work has shown that when alanine was substituted at positions 14, 35, 182, 232 and 465, no significant increase in the half-life at 37° C. was observed for 4 of the 5 mutants, which are F14A, L35A, V182A and F465A (Tisi et al., “Mutagenesis of solvent-exposed hydrophobic residues in firefly luciferase”, In Proceedings of the 11^{th} International Symposium on Bioluminescence and Chemiluminescence, pp 189-192). It is known that certain luciferase mutants with an increased half-life at temperatures of 37° C. and above have reduced bathochromic shifts (Tisi et al, 2002, “The basis of the bathochromic shift in the luciferase from Photinus pyralis”, In Bioluminescence and Chemiluminescence Progress and Current Applications, pp 57-60). Since reduction in the bathochromic shift is one mechanism for increasing the apparent low pH tolerance of a firefly luciferase, thermostable luciferase mutants can be expected to have a degree of increased tolerance to low pH.

Of key importance to this invention is that mutation of an amino acid at one or more of positions 14, 35, 182, 232 and 465 increases the specific activity of such firefly luciferase Mutants at pH values below the pH optimum of the wild-type enzyme, relative to the wild-type recombinant equivalent enzyme. Further, the corrected specific activities of the claimed mutants are higher than the wildtype recombinant equivalent enzyme at pH values below the pH optimum of the wild-type enzyme, relative to the wild-type recombinant equivalent. Remarkably, the specific activity, or the corrected specific activity, of the claimed mutants is not deleteriously affected where the mutants are assayed at optimal pH for firefly luciferases. This is contrary to other luciferase mutants with ‘improved’ characteristics which demonstrate a decrease in specific activity under optimal conditions of pH relative to the wildtype recombinant equivalent enzyme.

This behaviour of the claimed mutants is surprising as only one of the aforementioned mutants having alanine substitutions at 232 demonstrated a significant increase in half-life at 37° C.

It is speculated that the increased low pH tolerance demonstrated by mutations at positions 14, 35, 182, 232 and 465 may result from a mechanism unconnected with thermostability. Thermostability has been demonstrated to be related to the bathochromic shift. This is particularly surprising as these amino acids are exclusively solvent-exposed residues that are i) not known to play a direct role in the enzymatic reaction and ii) are non-conserved between difference luciferases. It is even more surprising that, given the significantly increased pH tolerance at low pH obtained by combining all these mutations to give a luciferase mutant with five point mutations (referred to hereafter as ‘His-luc×5’ or ‘x5’), there seems to be no significant deleterious effect on the specific activity of the mutant luciferase at optimal pH for a firefly luciferase relative to the equivalent recombinant wild-type luciferase; we show this is also the case when ‘corrected specific activity’ is considered. It is further surprising, given the increased pH tolerance at pH values below optimal pH for a firefly luciferase, that neither any of the individual mutants nor the combination of mutants to give His-luc×5, substantially affect the fundamental kinetic constants of the enzyme with respect to the substrates ATP and luciferin at pH 7.8. We speculate that this is a result of only mutating residues that are non-conserved and solvent exposed, as such residues are expected to be less likely to adversely affect the bioluminescent process catalysed by the luciferase. This is in contrast to previous work that has not specifically targeted non-conserved, solvent exposed residues in firefly luciferase for mutation to give pH tolerance.

Preferably, the luciferase has a mutation at more than one amino acid selected from the group consisting of positions 14, 35, 182, 232 and 465, for example two, three, four or five mutations. In a preferred embodiment, the luciferase has a mutation of at least one amino acid selected from the group consisting of positions 14, 35, 182 and 465, where the numbering is according to the sequence of the luciferase from P. pyralis (SEQ ID NO:1—wild type P. pyralis). More preferably, all of amino acids 14, 35, 182, 232 and 465 are mutated. In a particularly preferred embodiment, the luciferase is from P. pyralis. Most preferably, the luciferase is from P. pyralis and all of amino acids F14, L35, V182, I232 and F465 are mutated.

The mutations can be made using standard methods known in the art, e.g., site-directed mutagenesis (see e.g., Sambrook et al., (2001), Molecular Cloning, Cold Spring Harbour Laboratory Press). Alternatively, the luciferase is a luciferase from another organism, such as from Luciola iningrelica, Luciola cruciata, Luciola lateralis, Hotaria paroula, Pyrophorus plagiophthalamus (Green-Luc GR), Pyrophorus plagiophthalamus (yellow-Green Luc YG), Pyrophorus plagiophthalarnus (Yellow-Luc YE), Pyrophorus plagiophthalamus (Orange-Luc OR), Lampyris noctiluca, Pyrocelia nayako, Photinus pennsylanvanica LY, Photinus pennsylanvanica J19, or Phrixothrix green (PVGR) or red (Php9), where the luciferase has a mutation at least one position equivalent to the P. pyralis amino acids 14, 35, 182, 232 and 464.

The sequences of all the various luciferases show that they are strongly conserved, having a significant degree of similarity between them. This means that corresponding regions among the enzyme sequences are readily determinable by examination of the sequences using sequence alignments to detect the most similar regions, although if necessary, commercially available software (e.g., “Bestfit” from the University of Wisconsin Genetics Computer Group; see Devereux et al., (1984) Nuc. Acid Res. 12, 387-395) can be used in order to determine corresponding regions or particular amino acids between the various sequences. Alternatively, or in addition, corresponding amino acids can be determined by reference to Ye et al., Biochim. Biophys. Acta, 1339 (1997) 39-52.

Sequence alignment techniques can be used to determine which amino acid positions are equivalent in luciferases from different organisms.

It is preferred that amino acid at position 35 and/or 232 is not mutated to an alanine residue. Preferably, none of the amino acids at positions 14, 35, 182, 232 and 465 are mutated to alanine residues.

One, two, three, four or all of the amino acids at these positions are preferably mutated to a hydrophilic residue. Hydrophilic residues include aspartic acid, glutamic acid, histidine, lysine, asparagine, glutamine, arginine, and serine. More preferably, one, two, three, four or all of the amino acids at these positions are mutated to a positively charged residue. Positively charged residues include arginine, lysine and histidine. Preferably, the mutation at one, two, three or all of positions 14, 182, 232 and 465 is to a positively charged residue. In a particularly preferred embodiment, the mutations are preferably to the following residues: F14R, L35Q, V182K, I232K and F465R.

It has been found that the specific activity of mutants (expressed as His₁₀-tag proteins for ease of purification) having positive charges at one or more, preferably four of these five positions, is increased relative to His₁₀-tag LucWT (His-lucWT)) at acidic pH, such as at pH 6.5 (there are no relative differences in specific activity between LucWT and His-lucWT between pH 6.4 and 9.0). Further, the corrected specific activity of mutants having positive charges at one or more, preferably four of these five positions, is increased relative to His₁₀-tag LucWT (His-lucWT)) at acidic pH, such as at pH 6.5. The fact that the ‘corrected specific activity’ of the claimed mutants is also higher than His-LucWT at pH 6.5, demonstrates that this effect is not simply due to changes in the colour of emitted light. The fact that mutations introducing positively charged residues onto His-lucWT should increase low pH tolerance is surprising because the isoelectric point (pI) of His-lucWT is 7.2. A protein's isoelectric point is the pH at which the protein has an equal number of positive and negative charges. When a protein is buffered in a solution at the same pH as its isoelectric point, the protein is generally expected to be more stable (i.e. have a greater half-life) than at higher or lower pH values. By mutating solvent-exposed residues to positively charged residues, the isoelectric point of the mutant is increased. It would therefore be expected that the protein would be less tolerant to acidic pH. However, the inventors have surprisingly found that by mutating these solvent-exposed residues to positively-charged residues, the opposite effect occurs and the luciferase becomes more tolerant to acidic pH.

In addition to the effect on the bathochromic shift, mutations at one or more these five positions have surprisingly been found to affect the corrected specific activity of the luciferase at low pH (pH 6.5). For example, luciferases having a mutation at one position selected from the group consisting of F14R, L35Q, V182K, I232K and F465R, showed an increase in total light output (i.e. corrected specific activity) relative to His-lucWT at pH 6.5 (see Table 1).

TABLE 1

| | Bioluminescent corrected specific activity | | | |
|---|---|---|---|---|
| | | | Relative | |
| | | | activity | Relative |
| Enzyme | | | of pH | activity of |
| (His-luc | pH 7.8 | pH 6.5 | 6.5 to pH 7.8 | mutant to WT |
| versions) | (×10³ RLU) | (×10³ RLU) | (%) | at pH 6.5 (%) |
| WT | 47.7 | 11.8 | 25 | 100 |
| F14R | 58.6 | 17.9 | 30 | 151 |
| L35Q | 55.0 | 13.0 | 24 | 110 |
| V182K | 58.0 | 16.8 | 29 | 142 |
| I232K | 48.8 | 15.5 | 32 | 131 |
| F465R | 52.0 | 18.9 | 36 | 160 |
| x5 | 48.6 | 23.4 | 48 | 198 |

Table 1. demonstrates that for each of the mutants the corrected specific activity (ie. the total relative light units emitted over all wavelengths per equivalent amount of luciferase per unit time) is greater for the mutants at pH 6.5 compared to the wild-type enzyme at pH 6.5

It has been found that the effect on corrected specific activity (at pH 6.5) of mutating the amino acids at all five positions is additive, such that the His-luc×5 mutant has close to twice the corrected specific activity at pH 6.5 compare to His-lucWT. Further, it has surprisingly been found that the combination of the five mutations together does not result in a decrease in the corrected specific activity at pH 7.8 (i.e. the pH optimum of His-lucWT/LucWT). It has also been found that mutations at each or all of the positions have no substantial effect on the fundamental kinetic constants of the enzyme with respect to the substrates ATP and luciferin at pH 7.8 (see Table 2 below). Thus, in a preferred embodiment, the luciferase has mutations at all of positions 14, 35, 182, 232 and 465. Preferably, the luciferase has the following five mutations: F14R, L35Q, V182K, I232K and F465R. With this particular mutant, the bathochromic shift is completely absent. Moreover, the total absolute light emitted at pH 6.5 (i.e. the corrected specific activity) is increased nearly two-fold relative to His-lucWT at pH 6.5. This increase in light is not solely due to the absence of bathochromic shift as the results shown in Table 1 are independent of the wavelength of the emitted light. Nor is the result simply a function of the mutant being more stable (i.e. having a greater half-life than His-LucWT), as the low pH tolerance of His-luc×5 is comparable with that of the ‘UltraGlow’ luciferase from Promega which has a considerably longer half-life at 45° C. than His-luc×5. Thus there is clearly not a direct, simple relationship between low pH tolerance and thermostability.

The novel His-luc×5 therefore has significant advantages over the previously described mutant luciferases as it offers significant pH tolerance and increased thermostability, but without a deleterious effect on specific activity or kinetic constants.

TABLE 2

| | Specific activity | Kₘ for LH₂ | Kₘ for ATP | K_{cat} |
|---|---|---|---|---|
| Enzyme | (RLU mg⁻¹)^{a} | (μM) | (μM) | (×10¹⁰ RLU s⁻¹)^{b} |
| LucWT | 3.1 ± 0.2 × 10⁶ | — | 66 ± 8 | 10.500 ± 0.008 × 10¹⁰ |
| His-lucWT | 2.1 ± 0.1 × 10⁶ | 14 ± 2 | 62 ± 3 | 7.2 ± 0.5 |
| His-lucF14R | 2.0 × 10⁶ | 19 | 64 | 6.8 |
| His-lucL35Q | 2.0 × 10⁶ | 15 | 63 | 6.9 |
| His-lucV182K | 2.2 × 10⁶ | 15 | 72 | 7.7 |
| His-lucI232K | 2.4 × 10⁶ | 15 | 72 | 8.5 |
| His-lucF465R | 1.9 × 10⁶ | 16 | 64 | 6.7 |
| His-lucx5 | 1.9 × 10⁶ | 16 | 76 | 6.5 |

Table 2 shows specific activities (un-corrected) and apparent kinetic properties of various luciferases. In this case specific activity was determined by injecting 0.1 M Tris-acetate, 10 mM MgSO₄, 2 mM EDTA, 200 μM LH₂, 1 mM ATP, pH 7.8 into wells containing 1.52 μg of enzyme. This was carried out at 23±2° C. in a final volume of 300 μl with PMT voltage set at 520 mV. The k_{cat} values were calculated from the data used to determine the Kₘ values for ATP. Errors shown represent one standard error.

The recombinant wild type luciferase from Photinus pyralis (LucWT) is known to be unstable at 37° C. (White, P. J. et al. (1996), “Improved thermostability of the North American firefly luciferase: saturation mutagenesis at position 354”. Biochem. J. 319, 343-350.). Mutation of one or more of residues 14, 35, 182, 232 and 465 also confers thermostability on the enzyme in that the half-life of the mutants is increased at 37° C. or higher relative to LucWT/His-lucWT. The method of the present invention is therefore preferably performed at a temperature below 55° C. Preferably, the method is performed in the range 20° C.-55° C., 35° C.-50° C., 35° C.-45° C., 35° C.-40° C. or 36° C.-41° C. Most preferably, the method is performed within the range 37° C.-40° C. or at 40° C.

In particular, the His:lucx5 luciferase embodied herein is much more stable than recombinant wild-type luciferase at 40° C. Thus in a preferred embodiment, the His-luc×5 luciferase is used in a method carried out at 40° C.

In a preferred embodiment, the invention provides the use of the luciferase, which is both pH tolerant and thermostable, in an in vivo medical imaging method.

In some embodiments, the luciferase contains mutations at only one or more of positions 14, 35, 182, 232 and 465 and all other amino acids are wild type residues. In alternative embodiments, in addition to mutations at one or more of these five positions, the luciferase contains mutations at other positions, but still retains its ability to catalyse the efficient transfer of chemical energy into light. For example, the luciferase may additionally comprise mutations at positions that increase the thermostability of the luciferase. Such positions are known in the art (for example see Patent no. AU2004202277; White, P. J. et al., 2002. “Novel in vivo reporters based on firefly luciferase” In Bioluminescence and Chemiluminescence Progress and Current Applications, pp 509-512) and would be readily available to the skilled person. For example, the luciferase may additionally comprise mutations at one or more of positions 105, 214, 215, 234, 295, 354, 357 and 420. In a most preferred embodiment, the luciferase comprises mutations at positions 14, 35, 182, 232 and 465 and positions 105, 214, 234, 295, 354, 357 and 420 (the “His-lucx12” mutant). Although the luciferase of the invention containing mutations at one or more of positions 14, 35, 182, 232 and 465 is already thermostable, the combination of these mutations with other mutations provides a luciferase that is both pH tolerant and is highly thermostable with a half-life at 55° C. of 15 minutes or more. Such a luciferase is particularly valuable in applications in which the temperature is not optimum for wild-type luciferase, and in which the pH fluctuates. Significantly, such a luciferase is not only highly thermostable and pH tolerant, but it retains a surprisingly high specific activity at pH 7.8 and room temperature. For example, the only other luciferase mutant with a half-life at 55° C. greater than 15 minutes is the ‘UltraGlow’ luciferase from Promega. UltraGlow has just one sixth the specific activity of His-lucx12 at room temperature pH 7.8 (Table 3). Further, UltraGlow has a far lower Km for ATP compared to LucWT, His-lucWT or His-luc×5. As a result, it has a significantly reduced dynamic range for the detection of ATP, thus making it inappropriate for assays requiring a greater dynamic range for ATP detection.

TABLE 3

| Enzyme | Intensity (RLU) |
|---|---|
| His-lucWT | 1111 ± 13.5 |
| His-lucx5 | 1190 ± 10.6 |
| His-lucx12 | 133.3 ± 14.8 |
| Promega UltraGlow | 22.99 |

Table 3 shows specific activities of His-lucWT, His-luc×5, His-lucx12 and Promega UltraGlow luciferase. Enzymes were assayed by manually mixing 20 μl of 0.42 μM enzyme solution with 180 μl of 0.1 M Tris-acetate pH 7.8, 10 mM MgSO₄, 2 mM EDTA, 1.11 mM ATP, 222 μM LH₂, 300 μM CoA. Bioluminescence emitted was integrated over 5 s using the luminometer. Quoted errors represent one standard error.

Thus the mutations described at position 14, 35, 182, 232 and 465 provide a basis luciferase mutant on which to add further mutations where effects on the specific activity of the luciferase may be reduced compared to using recombinant wild-type luciferase as the basis on which to add mutations.

Thus the invention also provides a luciferase that has a mutation at positions 14, 35, 182, 232 and 465 and one or more positions selected from the group consisting of 105, 214, 215, 234, 295, 354, 357 and 420, wherein the numbering is according to the sequence of the luciferase from P. pyralis (SEQ ID NO:1). Preferably, the luciferase according to the invention has mutations at positions 14, 35, 182, 232, 465, 105, 214, 234, 295, 354, 357 and 420, wherein the numbering is according to the sequence of the luciferase from P. pyralis (SEQ ID NO:1).

The invention also provides the use of the luciferase of the invention in in vivo and in vitro methods. For example, in vivo imaging methods using luciferase as a reporter gene (as described above) or in vitro methods in which a luciferase system is used to detect nucleic acid amplification through an ELIDA assay (for example see WO2004/062338 and PCT/GB2004/000127).

In particular, the invention provides the use of the luciferase of the invention in a bioluminescence assay. The His-lucx12 mutant is one of only two luciferase mutants (the other being ‘UltraGlow’ from Promega) that is capable of being used in a particular manifestation of the assay known as ‘Bioluminescent Assay for Real Time’ (BART), which is a method for measuring the extent of isothermal nucleic acid amplification reactions using bioluminescence as the reporting system (PCT/GB2004/000127). In common manifestations of the BART technology a luciferase is required to maintain bioluminescent activity at temperatures of up to 50° C., 55° C. or 60° C. for periods of up to 1 hour, or longer than 1 hour, and with temperature variations from less than 0° C. to up to 60° C.

Thus in a preferred embodiment, the luciferase of the invention is used in a method for determining the amount of template nucleic acid present in a sample which comprises the steps of: i) bringing into association with the sample all the components necessary for nucleic acid amplification, and all the components necessary for a bioluminescence assay for nucleic acid amplification and subsequently: ii) performing the nucleic acid amplification reaction; iii) monitoring the intensity of light output from the bioluminescence assay; and iv) determining the amount of template nucleic acid present in the sample. In a most preferred embodiment, the components brought into association in step i) comprise: a) a nucleic acid polymerase, b) the substrates for the nucleic acid polymerase, c) at least two primers, d) a thermostable luciferase, e) luciferin, f) an enzyme that converts PPi to ATP and g) any other required substrates or cofactors of the enzyme of part f). Preferably, the enzyme that converts PPi to ATP is ATP sulphurylase.

Aside from issues associated with thermal inactivation, various technologies/assays can be envisaged where a luciferase is required to tolerate changes in pH, or low pH, as well as being thermostable at temperatures at or in excess of 50° C. over periods of greater than 10 minutes.

The invention will now be illustrated further, by way of example only, with reference to the following figures in which:

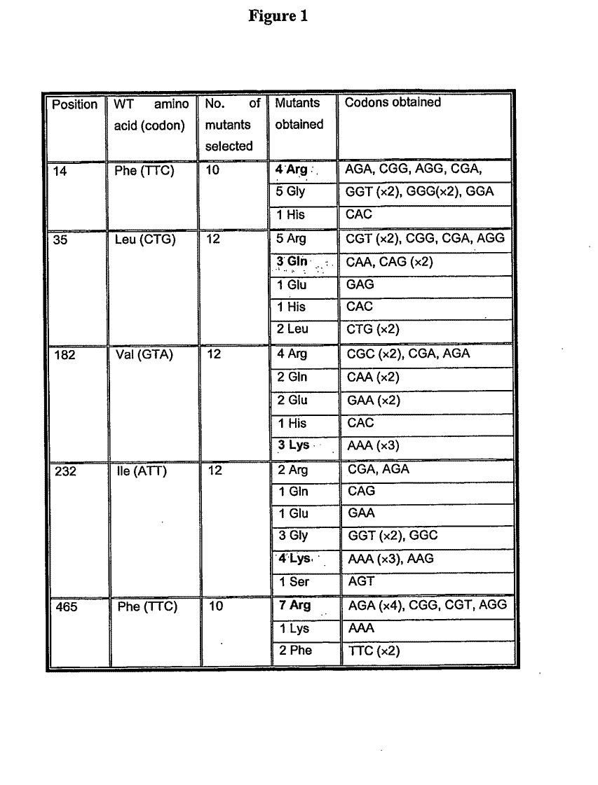

FIG. 1 shows a summary of the mutants obtained from random site-directed mutagenesis (“SDM”) carried out at positions F14, L35, V182, I232 and F465 of P. pyralis luciferase. Highlighted amino acids are those selected from the initial round of screening for each of these positions. It is seen that a large proportion of the selected mutants for all positions is either arginine or lysine;

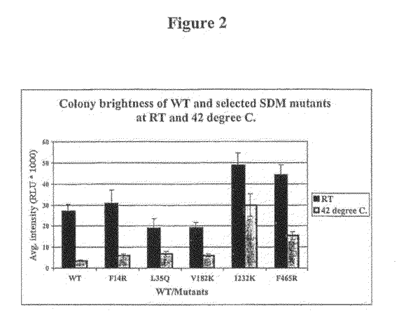

FIG. 2 shows a bar chart representation of the colony bioluminescence of colonies expressing WT luciferase and the five single point mutants integrated over a period of 5 s, at room temperature “RT” (approximately 23° C.) and 42° C. The average intensity for WT and/or mutant was taken from seven colonies expressing the same luciferase. Error bars represent one standard error. It is seen that I232K and F465R are both brighter than WT luciferase in the RT screen. However, in the 42° C. screen, all of the mutants are more apparently thermostable relative to WT with I232K being the most apparently thermostable;

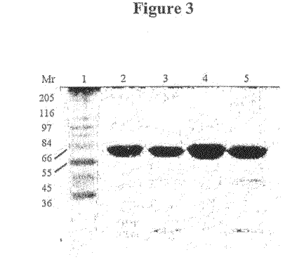

FIG. 3 shows SDS-PAGE (10%) analysis of the purity of Promega recombinant luciferase (Prluc, which is equivalent to LucWT) and His-lucWT. Lane 1—protein marker; lanes 2 & 4-5 & 10 μg of His-lucWT respectively; lanes 3 & 5-5 & 10 μg of Prluc respectively. Molecular weight of His-lucWT and Prluc are ˜63 kDa and ˜61 kDa respectively;

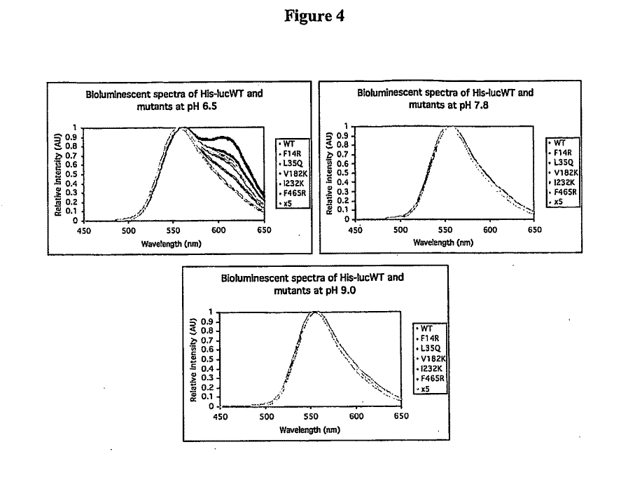

FIG. 4 shows normalised bioluminescent spectra of His-lucWT and mutants at, pH 6.5, 7.8 and 9.0. For each of the mutants and His-lucWT, 0.31 nmoles of enzyme was assayed with 1 ml of 0.1 M Tris-acetate, 10 mM MgSO₄, 2 mM EDTA, 200 μM LH₂, 1 mM ATP, 270 μM CoA, 2 mM DTT at 23±2° C. The bioluminescent spectra were recorded at 45 s after the initiation of the reaction over a period of 1 min;

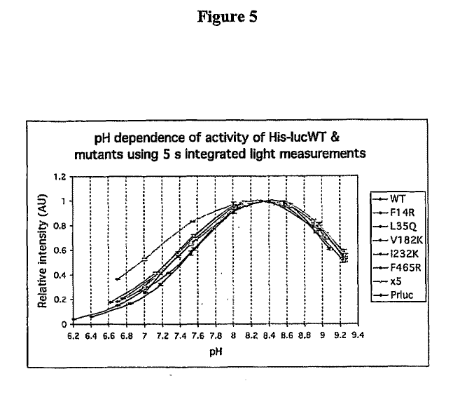

FIG. 5 shows a plot of relative bioluminescent intensity versus pH for His-lucWT and mutant luciferases. For His-lucWT and each of the mutants, 20 μl of 0.42 μM enzyme solution was assayed manually by mixing with 180 μl of 0.1 M Tris-acetate, 10 mM MgSO₄, 2 mM EDTA, 1.11 mM ATP, 222 μM LH₂ and 300 μM CoA. Bioluminescence emitted was integrated over 5 s using the luminometer. Error bars represent one standard error within triplicate measurements;

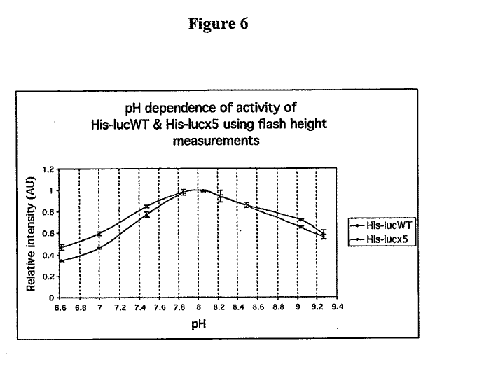

FIG. 6 shows a plot of relative intensity versus pH for the enzymes indicated. The amount of enzyme and substrates used were the same as described for FIG. 5 except that the substrates were injected into wells containing the enzyme in the luminometer. Flash height measurements were recorded. Error bars represent one standard error within triplicate measurements;

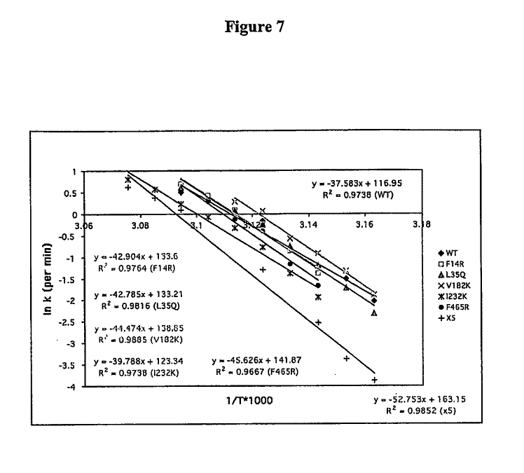

FIG. 7 shows an Arrhenius plot showing the dependence of rates of inactivation on temperature for WT and mutants;

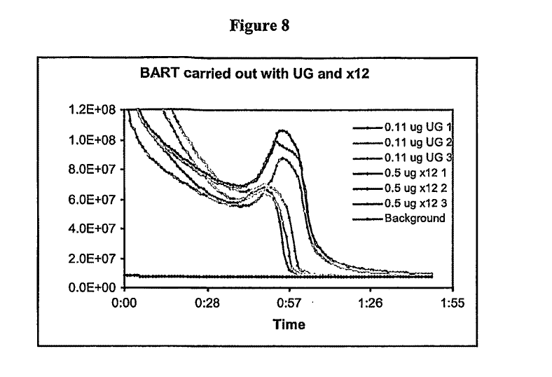

FIG. 8 shows the result of BART (an ELIDA-based assay according to patent PCT/GB2004/000127) using UltraGlow luciferase (Promega) and His-lucx12;

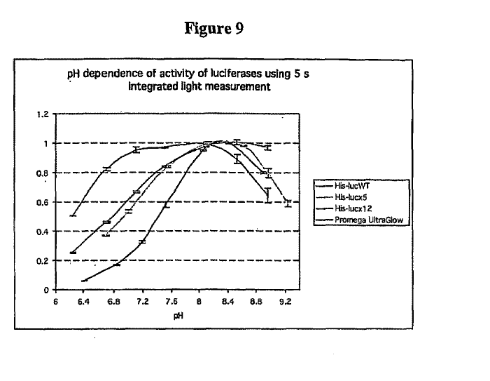

FIG. 9 shows a plot of relative intensity versus pH for His-lucWT, His-luc×5, His-lucx12 and Promega UltraGlow luciferase. 20 μl of 0.42 μM enzyme solution was assayed manually by mixing with 180 μl of 0.1 M Tris-acetate, 10 mM MgSO₄, 2 mM EDTA, 1.11 mM ATP, 222 μM LH₂ and 300 μM CoA. Bioluminescence emitted was integrated over 5 s using the luminometer. Error bars represent one standard error within triplicate measurements; and

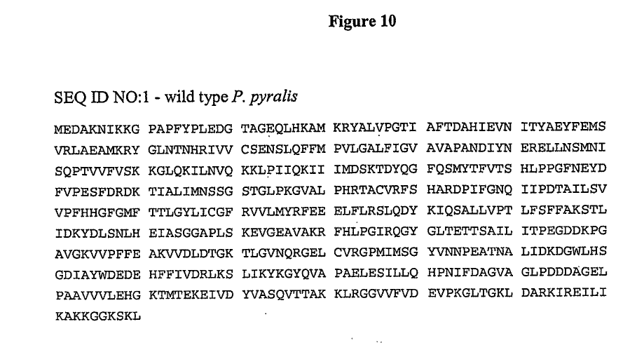

FIG. 10 shows the sequence of the luciferase from P. pyralis (SEQ ID NO:1).

### MATERIALS AND METHODS

### Materials

D-luciferin (LH₂) potassium salt was obtained from Europa Bioproducts (Ely, Cambridge, UK); EDTA-free protease cocktail inhibitor was from Roche Diagnostics GmbH; benzonase nuclease and Ni-NTA His.Bind resin were from Novagen. All other chemicals and reagents used were from Sigma-Aldrich Company Ltd., Fisher Scientific or Melford Laboratories Ltd. unless specified otherwise. E. coli strain XL2-Blue ultra-competent cells (Stratagene) were used as cloning hosts for the generation and selection of mutants from site-directed mutagenesis (SDM). For the over-expression of Histo-tag recombinant wild-type (His-lucWT) and mutant luciferases, BL21(DE3)pLysS (Novagen) was used. Plasmid pPW601L, derived from pPW601a (White, P. J. et al., (1996), “Improved thermostability of the North American firefly luciferase: saturation mutagenesis at position 354”, Biochem. J. 319, 343-350), with additional cloning sites, encoding for the WT P. pyralis luciferase gene was used in the random SDM experiments. Plasmid pET16b (Novagen) was used for the expression of N-terminal His₁₀-tagged luciferases and pET16b-luc was obtained by ligating the WT P. pyralis luciferase gene (E.C. 1.13.12.7) into pET16b.

### Site-Directed Mutagenesis, Screening and Selection of Mutants

Selective random site-directed mutagenesis (SDM) was carried out using the QuikChange™ Site-Directed Mutagenesis Kit (Stratagene) according to the manufacturer's protocol. Hot-start reactions each consisting of 18 cycles were carried out. Plasmid pPW601L was subjected to five rounds of mutagenesis with each using a pair of the following partially degenerate mutagenic primers: 5′-GGC CCG GCa CCA (CAG)(AG)(N) TAT CCT CTA GAG G-3′ and 5′-CCT CTA GAG GAT A(N)(CT) (CTG)TG GtG CCG GGC C-3′ (Hae II) for F14X; 5′-GGC TAT GAA GcG cTA CGC C(CAG)(AG) (N)GT TCC TGG-3′ and 5′-CCA GGA AC(N) (CT)(CTG)G GCG TAg CgC TTC ATA GCC-3′ (Hae II) for L35X; 5′-GAA TAC GAT TTT (CAG)(AG)(N) CCA GAa agC TTT GAT CG-3′ and 5′-CGA TCA AAG ctt TCT GG(N) (CT)(CTG)A AAA TCG TAT TC-3′ (Hind III) for V182X; 5′-GGC AAT CAA ATC(CAG)(AG)(N)CCG GAT ACT GCG-3′ and 5′-CGC AGT ATC CGG (N)(CT)(CTG) GAT TTG ATT GCC-3′ for I232X and 5′-CCC CAA CAT C(CAG)(AG) (N)GA CGC GGG CGT GGC AGG-3′ and 5′-CCT GCC ACG CCC GCG TC(N) (CT)(CTG)G ATG TTG GGG-3′ for F465X (lower case represents silent changes to modify a screening endonuclease site, the boldface type represents the mutated codon, and parentheses indicate the screening endonuclease used). Resultant mutants were screened and selected for brightness and apparent thermo-stability by imaging for light emission, using a CCD camera (Syngene Optics), from colonies at room temperature, and after incubation at 42° C., using an in vivo colony screen (Wood, K. V. and M. DeLuca (1987), “Photographic detection of luminescence in Eschericheria coli containing the gene for firefly luciferase”, Anal. Biochem. 161, 501-507). Colonies grown overnight at 37° C. were lifted onto a nylon membrane (Hybond N, Amersham) and these were assayed for light emission by spraying the colonies with 0.1 M citrate, 1 mM D-LH₂, pH 5.0. For each position, 80 random colonies were screened in the first round, resulting in the selection of between 10 and 12 mutants for the second round of screening, which were all sequenced (Department of Biochemistry, University of Cambridge).

The desired point mutant for each position was generated by SDM on pET16b-luc using the following primers: 5′-GGC CCG GCa CCA CGC TAT CCT CTA GAG G-3′ and 5′-CCT CTA GAG GAT AGC GTG GtG CCG GGC C-3′ (Hae II) for F14R; 5′-GGC TAT GAA GAG ATA CGC CCC GGT TCC TGG-3′ and 5′-CCA GGA ACC TGG GCG TAT CTC TTC ATA GCC-3′ for L35Q; 5′-GAA TAC GAT TTT AAA CCA GAa agC TTT GAT CG-3′ and 5′-CGA TCA AAG ctt TCT GGT TTA AAA TCG TAT TC-3′ (Hind III) for V182K; 5′-CGC AcG CCA GAG ATC CTA TTT TTG GCA ATC AAA TCA AAC CGG-3′ and 5′-CCG GTT TGA TTT GAT TGC CAA AAA TAG GAT CTC TGG CgT GCG-3′ (Sph I) for I232K and 5′-CCC CAA CAT CCG CGA CGC cGG CGT GGC AGG-3′ and 5′-CCT GCC ACG CCg GCG TCG COG ATG TTG GGG-3′ (Bgl I) for F465R (highlighted bases were as explained above). Plasmid pET16b-luc×5 was constructed by building one mutation upon another until all five mutations (F14R, L3SQ, V182K, I232K & F465R) were present in a single copy of the luciferase gene. The luciferase expressed from this construct is referred to as His-luc×5. Primer synthesis were carried out at facilities in the Department of Biochemistry and DNA sequencing was carried out by the sequencing facility at the Department of Genetics, both within the University of Cambridge.

### Expression and Purification of His₁₀-Tag Luciferases

His-lucWT and mutants were expressed from pET16b-luc in BL21(DE3)pLysS hosts. Cultures of 400 ml were grown in LB medium supplemented with 100 μg ml⁻¹ carbenicillin and 50 μg ml⁻¹ chloramphenicol in 2 L flasks at RT of ˜23° C. till an OD_{600 nm} of 0.8-0.9 AU is reached. Cultures were then induced with a final concentration of 1 mM IPTG for 6-8 h at the same temperature after which cells were harvested by centrifugation at 4° C. and stored overnight at −80° C. Cell pellets were resuspended in Lysis Buffer, which consists of Buffer A supplemented with 2% Triton X-100 (v/v) and 20 mM imidazole. (Buffer A comprised of 10 mM phosphate, 2.7 mM KCl, 0.3 M NaCl, 10 mM β-mercaptoethanol, 20% glycerol (v/v), 1×EDTA-free protease cocktail inhibitor (Roche Diagnostics GmbH), pH 8.0.) 5 ml of LB was used per gram wet weight cell. Benzonase nuclease (Novagen) was added to a final concentration of 125 Units g⁻¹ wet weight cell. Crude cell extract was obtained by centrifugation at 20 000 g, 4° C. for 1 h.

His-lucWT and mutants were then purified using Ni-NTA agarose (Novagen) affinity chromatography by loading the crude cell extract onto a chromatography column packed with Ni-NTA resin (1.5 cm diameter; 2.5 ml bed volume) at 4° C. Non-specifically bound proteins were removed with 4 column volumes of Buffer A containing 50 mM imidazole and the luciferases were eluted with 2.5 ml fractions of Buffer A containing 200 mM or 300 mM imidazole. Fractions of purified luciferases selected for further analysis consisted of fractions with the highest luciferase activity and purity based on activity measurement and SDS-PAGE analysis (Laemmli, U. K. (1970). Cleavage of structural proteins during the assembly of the bead of bacteriophage T4. Nature 227, 680-685) respectively. His-lucWT was also checked for purity by amino acid analysis carried out at facilities provided by the Department of Biochemistry, University of Cambridge. Each fraction of purified luciferase selected for further analysis was desalted on a PD-10 column (Pharmacia) into 3.5 ml of Storage Buffer, which comprises of 0.1 M Tris-acetate, 10 mM MgSO₄, 2 mM EDTA, 10% glycerol (v/v), 2 mM DTT, pH 7.8. These were stored in 50 or 100 μl aliquots at ±80° C. Total protein concentrations were estimated using the method of Bradford (Bradford, M. M. (1976). A rapid and sensitive method for the quantification of microgram quantities of protein utilising the principle of protein-dye binding. Anal. Biochem. 72, 248-254), using the Coomassie® Protein Assay Reagent Kit from Pierce according to the manufacturer's protocol, with BSA as the standard.

### Luciferase Activity Assays

### Dilution of Enzyme and Activity Assay Buffer

Luciferase enzymes were diluted from the purified enzyme stock solution into 0.1 M Tris-acetate, 10 mM MgSO₄, 2 mM EDTA, 2 mM DTT, pH 7.8 at 23° C.±2° C. to obtain the required concentration. In some experiments, a final concentration of either 2 or 10% glycerol (v/v) was added to the diluted enzyme solution. For the thermal inactivation assays, the enzymes were diluted into phosphate buffer and are described separately in section 2.16. The activity assay buffer consists of 0.1 M Tris-acetate, 10 mM MgSO₄, 2 mM EDTA, pH 7.8, at 23° C.±2° C., with varying concentrations of ATP and LH₂. In some experiments, CoA was added to the activity assay buffer. The exact concentrations of ATP, LH₂ and CoA are defined in each experiment. The volume of substrate-containing buffer and enzyme solution used varied and are specified in each experiment.

### Methods of Luciferase Activity Measurement

Luciferase activity was measured by injection or manual mixing of the assay buffer into wells of a 96 well microtiter plate (Labsystems) containing the luciferase sample. This was carried out on a Labsystems Luminoskan Ascent luminometer. Measurements of either flash height (i.e. intensity maximum, Iₘₐₓ) or integrated light intensities both reflect luciferase activity. These were recorded in RLU. Photo-multiplier tube (PMT) voltage varies and is specified in each experiment. All activity measurements were carried out at 23° C.±2° C.

### Determination of Kinetic Constants, Bioluminescent Spectra and “Corrected Specific Activity”

Procedures for the determination of Kₘ values for ATP and LH₂ were as described previously (Tisi, L. C. et al., (2002), “Development of a thermostable firefly luciferase”, Anal. Claim. Acta 457, 115-123). The bioluminescent spectra of His-lucWT and mutants were obtained by mixing assaying buffer consisting of 1 ml of 0.1 M Tris-acetate, 10 mM MgSO₄, 2 mM EDTA, 200 μM LH₂, 1 mM ATP, 270 μM CoA, 2 mM DTT with 0.31 nmoles of luciferase in a 1 ml plastic cuvette. These were carried out using assaying solutions at pH 6.5, 7.8 and 9.0 spectra were recorded using a Perkin Elmer LS50B spectrophotometer with a dead time of 30 s. All spectra were recorded from 450 nm to 650 nm, with a slit width of 10 nm and were scanned at 200 nm min⁻¹ with a PMT voltage of 900 mV. All spectra presented were corrected for the baseline and sensitivity of the PMT. Correction for the sensitivity of the PMT was carried out by calibration with lucifer-yellow and using the known spectra from Molecular Probe (Oregon). These experiments were subject to pH accuracy of ±0.05 unit, timing accuracy of ±5 s and temperatures of 23±2° C. Integration of the area under the bioluminescent spectra was used to obtain the “corrected specific activity” of luciferases, that is, the total amount of light (measured in relative light units) emitted per second per equivalent amount of luciferase (by mass) under saturating amounts

### Dependence of Bioluminescent Specific Activity on pH

For the determination of specific activity using integrated light measurements, 180 μl of assaying buffer consisting of 0.1 M Tris-acetate, 10 mM MgSO₄, 2 mM EDTA, 1.11 mM ATP, 222 μM LH₂, 300 μM CoA was mixed with 20 μl of 0.42 μM enzyme solution. This was carried out over the range of pH values between 6.0 and 9.5 with measurements at each pH carried out in triplicate. The enzyme was diluted in 0.1 M Tris-acetate, 10 mM MgSO₄, 2 mM EDTA, 2 mM DTT at pH 7.8. Light emitted was integrated for 5 s using the luminometer. These were carried out at the PMT voltage of 550 mV for all luciferases except Promega UltraGlow luciferase, which was measured at the PMT voltage of 700 mV so that reliable measurements could be made. The lag time between initiation of the reaction and recording of light emission is ˜5 s. For determination of specific activity using flash height measurements, the solutions used were the same as that described for the integrated light measurements, except that CoA was omitted. Enzymes in this instance were diluted in 0.1 M Tris-acetate, 10 mM MgSO₄, 2 mM EDTA, 2 mM DTT, 2% glycerol (v/v), at pH 7.8. 180 μl of the assaying solution at different pH was injected into wells containing 20 μl of 0.42 μM enzyme solution. Triplicate measurements at each pH were carried out and the flash height (Iₘₐₓ) was recorded at a PMT voltage of 550 mV.

### Thermal Inactivation Assays of His-lucWT and His-luc×5

The methodology of thermal inactivation assays was as described previously (Tisi, L. C. et al., (2002), “Development of a thermostable firefly luciferase”, Anal. Chim. Acta 457, 115-123) except that luciferases were diluted into 50 mM potassium phosphate, 10% glycerol (v/v), 2 mM DTT, pH 7.8. Temperatures assayed ranged between 37 and 50° C. for varying lengths of time up to 120 min. Bioluminescent activity was determined using flash intensity measurements by injecting 100 μl of 0.1 M Tris-acetate, 10 mM MgSO₄, 2 mM EDTA, 1.05 mM ATP, 210 μM LH₂ into wells containing 5 μl of 0.2 μM enzyme solution. PMT voltage was set at 760 mV for all the measurements. Rates of inactivation were calculated from sets of data that exhibited an apparently first-order reaction and these were used to construct the Arrhenius plot.

### EXAMPLES

### 1. Mutagenesis, Screening and Selection of Luciferase Mutants

Positions F14, L35, V182, I232 and F465 in Photinus pyralis luciferase were chosen for mutagenesis as have been previously shown to be amenable to changes without affecting the catalytic activity (Tisi, L. C. et al., (2001), “Mutagenesis of solvent-exposed hydrophobic residues in firefly luciferase”, In. Case, J. F., et al (Eds.). Proceedings of the 11^{th} International Symposium on Bioluminescence and Chemiluminescence, pp. 189-192, World Scientific, Singapore). These were mutagenised randomly to eight hydrophilic amino acids using semi-random SDM. Resulting colonies were screen at room temperature and after they have been incubated at 42° C., which facilitated the selection of potentially thermo-stable mutants. From the first round of screening, between 10 and 12 mutants were selected and sequenced (FIG. 1). From these, a second round of screening allowed the selection of the brightest and/or most apparently thermo-stable mutant for each position, which were found to be F14R, L35Q, V182K, I232K and F465R (FIG. 2).

Of the five mutants selected, it is noted that four had a positively charged amino acid substituted. Further more, at each position, the proportion of mutants with similar substitution selected from the first screen is greater than that expected from the number of codons encoding for these amino acids. This suggests the favourable addition of a positively charged group at these sites. Previous observations showed that when colonies of E. coli have been assayed for light emission in the same way, red bioluminescence was observed (unpublished data). Thus, the selection of brighter mutants in vivo might also select for mutants that lack the bathochromic shift due to the lower sensitivity of the CCD camera used to red light.

### 2. Construction, Expression and Purification of Luciferase Mutants

Using SDM, luciferases having one mutation selected from the group consisting of F14R, L35Q, V182K, I232K and F465R were individually constructed on pET16b-luc which expresses the protein with an N-terminal Histo-tag. A ×5 mutant, which comprises all five of these mutations, was also constructed on pET16b-luc. These, together with wild-type luciferase of P. pyralis (His-lucWT), were expressed in BL21(DE3)pLysS and purified using Ni-NTA resin by affinity chromatography as detailed in materials and methods. Fractions with purity of >90%, determined by amino acid analysis, were obtained and used for subsequent characterisation. His-lucWT was shown to be purer than recombinant luciferase obtained from Promega when analysed using SDS-PAGE (FIG. 3). However, the specific activity of His-lucWT is only ˜68% of that of Promega recombinant luciferase (Table 2). This agrees with previous findings, which showed His-tag luciferase to have a lower specific activity than that of recombinant WT luciferase (Branchini, B. R. et al., (2000), “The role of lysine 529, a conserved residue of the acyl-adenylate-forming enzyme superfamily, in firefly luciferase”. Biochemistry 39, 5433-5440; Michel, P., et al (2001), “Expression and purification of polyhistidine-tagged firefly luciferase in insect cells—a potential alternative for process scale-up”, J. Biotechnol. 85, 49-56). Significantly, the His-luc mutants show very similar specific activity to His-lucWT at pH 7.8.

### 3. Kinetic Analysis of Luciferase Mutants

Dependence of D-LH₂ and ATP on bioluminescence activity of the mutants and His-lucWT were investigated. Kₘ and Kcat values of His-lucWT and mutants for both of these substrates showed, little difference (Table 2). The rise times for these reactions were also observed to be the same, suggesting no change in kinetics under the same assaying conditions. This is consistent with the unchanged specific activity observed at pH 7.8 (the same pH at which the kinetic analysis was performed; see Table 1).

### 4. pH Dependencies of Colour of Bioluminescence and “Corrected Specific Activity” of Luciferase Mutants

The colour of bioluminescence for these mutants at three different pH values was also determined. These were carried out, under the condition of saturating substrates, in the presence of CoA, over a period of one min, using equal amount of enzyme for each reaction. The normalised bioluminescent spectra of His-lucWT and mutants (FIG. 4) showed similar spectral profiles, with a single maximum at 556 nm, at pH 7.8 and 9.0. At pH 6.5, a subsidiary maximum at about 610 nm is observed for His-lucWT. This appears to be due to the formation of a red-emitting species. It is seen that the contribution of this second species is reduced for all mutants, with the exception of L35Q, the only uncharged mutation. For His-luc×5, the subsidiary maximum at about 610 nm is completely absent. The absolute light emitted at pH 6.5 (i.e. the corrected specific activity) showed a nearly two-fold increase for His-luc×5 relative to His-lucWT, further all the single point mutants, showed increases in light emission at pH 6.5 relative to His-lucWT (Table 1). As these mutations are 1) not thought to be part of the active site, 2) non-conserved within the family of firefly luciferases and 3) do not show significant changes in their Kₘ values for both D-LH₂ and ATP, it is therefore unexpected that the mutants should have increased corrected specific activity, relative to His-lucWT, at pH 6.5.

The bioluminescent specific activity of luciferase mutants over a pH range of 6.2-9.4 was measured by using the light integrated over 5 s upon the initiation of the reaction. The His-luc×5 mutant showed a significantly higher level of bioluminescence in the acid range of pH values (FIG. 5). Specific activity over the same pH range was also measured by monitoring flash heights. It is seen that the optimal pH for both His-lucWT and His-luc×5 is ˜pH 8.0 (FIG. 6), which agreed with that reported in the literature. Thus, altering the five surface residues at the selected positions did not change the optimal pH for the bioluminescent reaction. The raised optimal pH seen in FIG. 5 is a result of an artefact due to the integration of light emitted after the flash, which is not a true reflection of total luciferase activity.

The precise molecular mechanism by which the mutations making up His-luc×5 are able to have i) increased specific activity (corrected or not) at pH 6.5 relative to His-lucWT ii) reduction in bathochromic shifts iii) increased thermostability and iv) similar specific activity (corrected or not) as His-lucWT at optimal pH either as point mutations or collectively, is not fully understood. It is clear that these properties are not simply a function of thermostability. For example, the UltraGlow enzyme from Promega is more stable than any luciferase mutant derived from Photinus pyralis, yet (as FIG. 9 demonstrates) the relative specific activity at pH values below pH 8.00 is similar to the far less stable His-luc×5. Further, whilst the UltraGlow enzyme from Promega is more stable than His-lucx12, FIG. 9 shows that the latter has increased tolerance to low pH than the former.

### 5. Thermal Inactivation of Luciferase Mutants

Rates of thermal inactivation of luciferase mutants were obtained by incubating aliquots of enzyme solutions at temperatures between 43 and 52° C.

Arrhenius plots of thermal inactivation rates shot that mutant I232K has a similar gradient to that of His-lucWT whereas His-luc×5 showed the largest increase in gradient (FIG. 7). According to transition state theory, the activation enthalpy (ΔH^{‡}) can be calculated from the gradient of the Arrhenius plot whereas the ln k-intercept gives an indication of the activation entropy (ΔS^{‡}). All the mutants thus show an increase in both the ΔH^{‡} and ΔS^{‡}, consistent with stabilisation by electrostatic interactions, where there is an entropic cost due to the fixing of the orientation of the side-chains. ΔH^{‡} values for His-lucWT and His-luc×5 are calculated to be +310 kJ mol⁻¹ and +440 kJ mol⁻¹ respectively. The ΔΔS^{‡} between His-lucWT and His-luc×5, calculated from the ln k-intercepts, is found to be +380 JK⁻¹ mol⁻¹. At 40° C., the ΔΔG^{‡} between His-lucWT and His-luc×5 is calculated to be +7 kJ mol⁻¹, so His-luc×5 is much more stable at this temperature.

In FIG. 9 is seen the normalised (with respect to the maximal specific activity observed) pH dependent specific activity profiles for different luciferases. The normalised activity profile of His-luc×5 is very similar to that of Ultra-Glow luciferase at pH values less than pH 8.4. This is despite the fact that His-luc×5 is far less thermostable than UltraGlow luciferase. This, again, emphasises that thermostability and low pH tolerance are not necessarily proportionately related.

FIG. 9 also shows that His-lucx12 has the broadest pH tolerance at pH values less than 7.7 of any other luciferases shown herein. As a result, the His-lucx12 is not only extremely thermostable, (as demonstrated by its ability to be used as an alternative to UltraGlow in BART reactions; see FIG. 8) but has far greater tolerance to low pH than any other mutant described herein, including the more thermostable UltraGlow enzyme from Promega. The His-lucx12 thus takes advantage of the 5 mutations disclosed in this invention to offer a luciferase that is extremely tolerant to low pH relative to other luciferases, as well as being highly thermostable. FIG. 8 demonstrates the utility of such a mutant luciferase in the aforementioned BART assay.

1. The use of a luciferase that has a mutation of at least one amino acid selected from the group consisting of positions 14, 35, 182, 232 and 465, where the numbering is according to the sequence of the luciferase from P. pyralis (SEQ ID NO:1) in a method that is performed at a pH below the optimal pH for the wild-type luciferase during at least part of the time period over which bioluminescence measurements are taken, wherein the specific activity of the mutant luciferase is higher than the specific activity of wild-type at the pH at which the method is carried out.

2. A use according to claim 1, wherein the method is performed at below pH 7.7 during at least part of the time period over which bioluminescence measurements are taken.

3. A use according to claim 1, wherein the pH is within the range of 6.0 to 7.6 during at least part of the time period over which bioluminescence measurements are taken.

4. A use according to claim 1, wherein the pH is within the range of 6.0 to 7.0 during at least part of the time period over which bioluminescence measurements are taken.

5. A use according to claim 4, wherein the pH is around 6.5 during at least part of the time period over which bioluminescence measurements are taken.

6. A use according to claim 1, wherein at least one bioluminescence measurement is taken when the pH is below the optimal pH for wild-type luciferase.

7. A use according to claim 6, wherein all of the bioluminescence measurements are taken when the pH is below the optimal pH for wild-type luciferase.

8. A use according to claim 1, wherein the pH remains constant throughout the method.

9. A use according to claim 1, wherein the pH fluctuates during the method.

10. A use according to claim 1, wherein the luciferase is from P. pyralis.

11. A use according to claim 1, wherein one or more amino acids are mutated to hydrophilic amino acids.

12. A use according to claim 1, wherein one or more amino acids are mutated to positively charged amino acids.

13. A use according to claim 1, wherein the mutations are selected from the group consisting of F14R, L35Q, V182K, I232K and F465R.

14. A use according to claim 1, wherein the luciferase has mutations at all of positions 14, 35, 182, 232 and 465.

15. A use according to claim 1, wherein the method is performed within a temperature range of 20° C.-55° C.

16. A use according to claim 1, wherein the method is performed within a temperature range of 36° C.-41° C.

17. A use according to claim 1, wherein the luciferase contains mutations at only one or more of positions 14, 35, 182, 232 and 465 and all other amino acids are wild type residues.

18. A use according to claim 1, wherein in addition to mutations at one or more of positions 14, 35, 182, 232 and 465, the luciferase contains mutations at one or more positions selected from the group consisting of: 105, 214, 215, 295, 234, 354, 357 and 420.

19. A use according to claim 18, wherein in addition to mutations at one or more of positions 14, 35, 182, 232 and 465, the luciferase contains mutations at all of positions 105, 214, 234, 295, 354, 357 and 420.

20. A use according to claim 1, wherein the method is carried out in vitro.

21. A use according to claim 1, wherein the method is carried out in vivo.

22. A use according to claim 1, wherein the method is a method of in vivo imaging of one or more tissues or cells of a live organism.

23. A use according to claim 1, wherein the luciferase is used in a bioluminescent assay to detect nucleic acid amplification, wherein the amplification reaction is performed at a temperature greater than 30° C.

24. A use according to claim 1, wherein the method is a diagnostic assay.

25. A luciferase that has a mutation at positions 14, 35, 182, 232 and 465 and one or more positions selected from the group consisting of 105, 214, 215, 234, 295, 354, 357 and 420, wherein the numbering is according to the sequence of the luciferase from P. pyralis (SEQ ID NO:1).

26. A luciferase according to claim 25, wherein the luciferase has mutations at positions 14, 35, 182, 232, 465, 105, 214, 234, 295, 354, 357 and 420, wherein the numbering is according to the sequence of the luciferase from P. pyralis (SEQ ID NO:1).

27. The use of the luciferase of claim 25 in a bioluminescence assay.

28. The use of claim 27, wherein the bioluminescence assay is a method for determining the amount of template nucleic acid present in a sample and comprises the steps of: i) bringing into association with the sample all the components necessary for nucleic acid amplification, and all the components necessary for a bioluminescence assay for nucleic acid amplification and subsequently: ii) performing the nucleic acid amplification reaction; iii) monitoring the intensity of light output from the bioluminescence assay; and iv) determining the amount of template nucleic acid present in the sample.

29. A kit comprising a luciferase as recited in claim 25.
